(19) United States
(12) Patent Application Publication
(10) Pub. No.: US 2008/0183242 A1
(43) Pub. Date: Jul. 31, 2008

(54) Electrical stimulation method for vision improvement

(75) Inventors: Yasuo Tano, Kobe-shi (JP); Hirokazu Sakaguchi, Minoh-shi (JP); Hugo Quiroz Mercado, Barrio del nino Jesus Coyoacan (MX); Eiji Yonezawa, Okazaki-shi (JP); Hiroyuki Kanda, Minoh-shi (JP)

Correspondence Address: OLIFF & BERRIDGE, PLC, P.O. BOX 320850, ALEXANDRIA, VA, 22320-4850, US

(73) Assignee: NIDEK CO., LTD., Gamagori-shi (JP)

(21) Appl. No.: 11/698,948

(22) Filed: Jan. 29, 2007

(51) Int. Cl. A61N 1/00

(52) U.S. Cl. 607/53

(57) ABSTRACT

A method for improving visual ability of even a patient's non-operative eye is provided. The electrical stimulation method for improving vision of patient's eyes comprises placing an electrode in one of patient's right and left eyes, and outputting an electrical stimulation pulse signal from the electrode placed in the patient's eye under a predetermined stimulation condition to stimulate cells constituting a retina or optic nerve, thereby improving vision of the other patient's eye with no electrode being placed.

### BACKGROUND OF THE INVENTION

1. Field of the Invention

The present invention relates to an electrical stimulation method for improving vision (sense of sight) by use of a stimulating electrode.

2. Description of Related Art

Retinitis pigmentosa, age-related macular degeneration, and others are likely to cause visual defects which may lead to vision loss. When light is irradiated to a retina of a human eye, normally, a light signal is converted in photoreceptor cells into an electrical signal. This electrical signal comes to a pulse signal in retinal ganglion cells and transmitted to a brain to acquire vision. When the retinitis pigmentosa, age-related macular degeneration, and others are caused, photoreceptor cells will decrease or become extinct. Thus, the light signal could not be converted into the electrical signal and hence a person could not obtain vision.

In recent years, various attempts have been proposed to recover vision of such blind persons. For example, U.S. Pat. No. 5,109,844 discloses an invention related to a visual restoration aiding apparatus utilizing a retina stimulating type implant. This visual restoration aiding apparatus is arranged such that an intraocular implantable device (an internal device) including a photodiode array (an image pickup device), electrodes, a signal processing circuit, and others is placed on or under a retina to pick up (receive) an image (light) in order to obtain vision. Further, JP-A-2004-181100 discloses a system arranged such that electrodes are stuck in an optic papilla (optic disc) of a patient's eye to electrically stimulate an optic nerve, thereby allowing a patient to perceive a photographed image captured by an imaging device located outside a body.

### SUMMARY OF THE INVENTION

Each of the aforementioned apparatuses is made as an attempt to artificially create external images by using an electrical stimulating signal output from the electrode, but not directed to improve visual ability itself of the patient. Further, each aforementioned apparatus is arranged to aid visual restoration of a patient's eye (an operative eye) in which the apparatus is implanted.

The present invention has an object to provide a method for improving even visual ability of a non-operative eye of a patient.

To achieve the above object, there is provided an electrical stimulation method for improving vision of patient's eyes, comprising the following steps: placing an electrode in one of the patient's eyes; and outputting an electrical stimulation pulse signal from the electrode placed in the patient's eye under a predetermined stimulation condition to stimulate cells constituting a retina or an optic nerve to improve vision of the other patient's eye with no electrode placed.

According to the present invention, it is possible to improve even visual ability of a non-operative eye of a patient.

### BRIEF DESCRIPTION OF THE DRAWINGS

FIG. 1 is a block diagram showing a schematic configuration of an electrical stimulation apparatus used in a present embodiment.

FIG. 2 is a view showing a configuration of a stimulation condition setting part.

FIG. 3 is a schematic view showing a placement state of electrodes and a horizontal sectional view of an entire eyeball.

FIG. 4 is a schematic view showing a placement state of the electrodes and a view showing an optic papilla and its surrounding area.

### DETAILED DESCRIPTION OF THE PREFERRED EMBODIMENTS

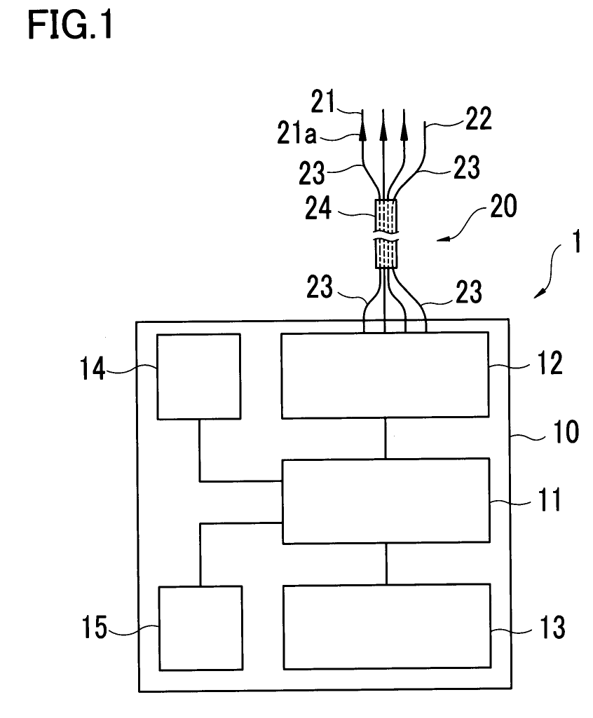

A detailed description of a preferred embodiment of the present invention will now be given referring to the drawings. FIG. 1 is a block diagram showing a schematic configuration of an electrical stimulation apparatus for vision improvement used in the present embodiment.

An electrical stimulation apparatus 1 comprises a stimulation pulse signal generating unit 10 and a stimulating electrode unit 20. The stimulation pulse signal generating unit 10 includes a control unit 11 such as a CPU, a pulse signal converting circuit 12, a stimulation condition setting part 13, a memory 14, and a display 15. These pulse signal converting circuit 12, stimulation condition setting part 13, memory 14, and display 15 are electrically connected to the control unit 11.

The pulse signal converting circuit 12 serves to generate a signal for electrical stimulation pulse for improving (restoring) vision based on stimulation conditions set in the stimulation condition setting part 13 and transmit the signal to the stimulating electrode unit 20. The stimulation condition setting part 13 includes an adjusting dial, various switches, and others for setting various stimulation conditions such as setting of output conditions of stimulation pulse signals and a combination of individual stimulation by stimulating electrodes 21 mentioned later and simultaneous stimulation by a plurality of electrodes. The display 15 displays various setting conditions.

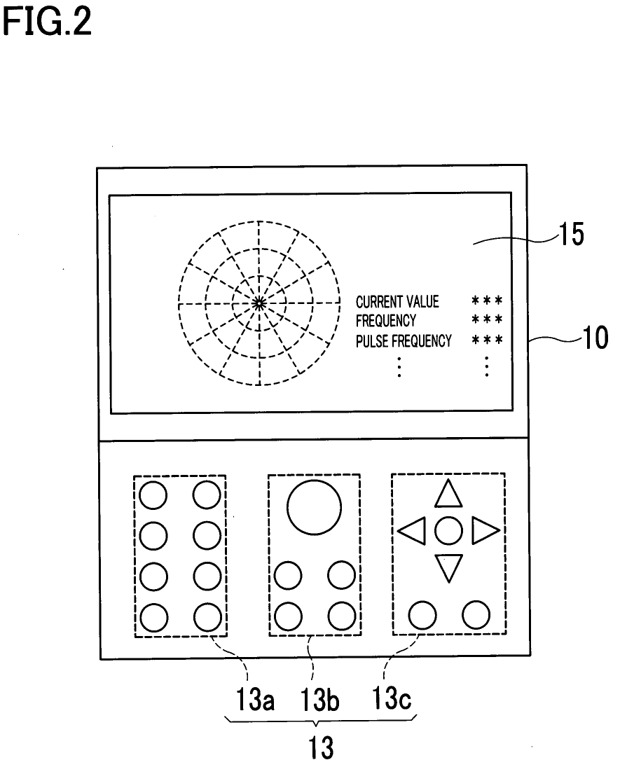

FIG. 2 is a view of a schematic configuration of the stimulation condition setting part 13 of the stimulation pulse signal generating unit 10. Numeral 13a denotes an output electrode specifying part, which includes switches for specifying electrodes to output stimulation pulse signals. Numeral 13b denotes an output condition setting part, which includes switches and the adjusting dial for setting output conditions of the electrical stimulation pulse signals to be output from the electrodes specified in the output electrode specifying part 13a. Numeral 13c denotes a phosphene position setting part, which includes switches for recording the positions in a visual field of a patient where phosphenes have been generated by the electrical stimulation pulse signal output from each stimulating electrode 21. Note that the memory 14 stores a plurality of stimulation conditions for each electrode set in the stimulation condition setting part 13.

The stimulation pulse signal generating unit 10 configured as above is preferably designed to have a size that allows a patient to carry.

The stimulating electrode unit 20 includes a plurality of stimulating electrodes 21 and an indifferent electrode 22. The stimulating electrode unit 20 is electrically connected to the stimulation pulse signal generating unit 10 through signal wires 23. A tip of each signal wire 23 may be formed as an electrode. At least one of the stimulating electrodes 21 is required only to be provided. Each stimulating electrode 21 is made of a conductive material superior in biocompatibility and corrosion resistance such as gold and platinum, and formed in a needle or another shape easy to stick in the optic papilla. Such electrode 21 preferably has a diameter that provides rigidity enabling to stick in the optic papilla. The diameter of each electrode 21 is preferably 10 μm to 200 μm, more preferably about 30 μm to about 100 μm. If the diameter of the electrode 21 is less than 10 μm, it is hard to stick in the optic papilla. If the diameter of the signal wire exceeds 200 μm, rigidity of the wire itself is increased, resulting in a difficulty in handling the tip of the signal wire 23 as an electrode. The length of such electrode 21 is required only to be so long as to conduct desired electrical stimulation to optic nerve fibers. The length of the electrode is appropriately determined based on an electrode material and a surface area of the electrode. The length of the electrode is preferably 200 μm to 2 mm, more preferably about 400 μm to about 1 mm.

The indifferent electrode 22 is made of the same material as the stimulating electrode 21. The electrode 21 and the indifferent electrode 22 are entirely coated excepting respective tips with a good biocompatible and insulating material such as polyimide and polyparaxylylene. Further, each signal wire 23 is entirely coated similarly with a good biocompatible and insulating material such as polyimide and polyparaxylylene.

Each electrode 21 is provided with a stopper portion 21a for preventing the electrode 21 stuck in the optic papilla from falling off. This stopper portion 21a is of a wedge shape and arranged at a position away from the tip toward the base end of the electrode 21 by a predetermined distance. The stopper portion 21a has to be provided at a portion of the electrode 21 that is coated with the aforementioned insulating material and that will not appear outside the optic papilla (inside a vitreous body) when the electrode 21 is stuck (implanted) in the optic papilla. The stopper portion 21a may be formed in such a manner that the aforementioned good-biocompatible and insulating coating material is processed into a wedge or another shape serving to prevent falling-off and joined to the coated portion of the electrode 21.

Further, each signal wire 23 may be formed with an electrode distinguishing part (mark) for allowing an operator to distinguish each of the electrodes 21 stuck in the optic papilla. This electrode distinguishing part is preferably formed on the signal wire 23 which appears outside the optic papilla (inside the vitreous body) when the electrode 21 is stuck in the optic papilla by a predetermined depth. This electrode distinguishing part is configured in such a manner that for example the coated portion (the coated layer) of the signal wire 23 is cut away by a predetermined amount in a direction of thickness by an excimer laser or the like to alternately form protrusions and recesses in an axial direction. The numbers of protrusions and recesses in each coated portion may vary from one electrode 21 to another, so that implanted electrodes can be distinguished individually, which assists setting by use of the output electrode specifying part 13a of the stimulation condition setting part 13.

The plurality of signal wires 23 is tied into a bundle by a tube 24 and therefore easy to handle. For the tube 24, a tube made of a material good in biocompatibility and superior in flexibility, such as silicone, is used. Note that each signal wire 23 connected to the electrode 21 or the indifferent electrode 22 is a strand wire made of a plurality of fine electric wires tied into a bundle, so that appropriate flexibility and rigidity and the like can be maintained in a balanced manner and handled with ease. The thickness (diameter) of such signal wire 23 is not always limited to the same thickness as the stimulating electrode 21 and is determined in consideration of the flexibility and rigidity and the like. Instead of providing the electrode and the signal wire as separate components, the tip of the signal wire 23 may be used as an electrode.

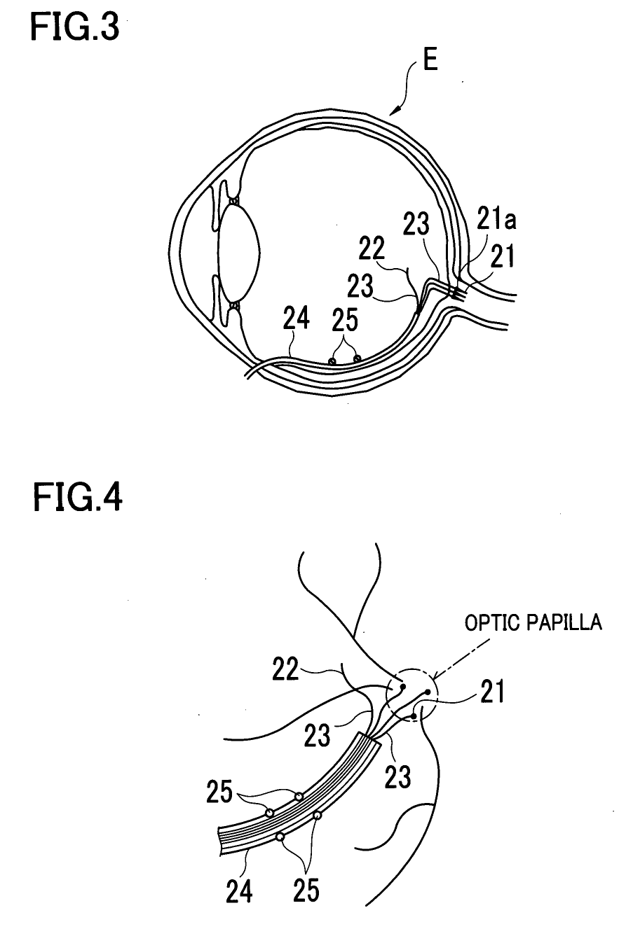

Those electrodes 21 and indifferent electrode 22 of the stimulating electrode unit 20 are previously implanted in a body (in an eye) of a patient by surgery. The electrodes 21 are implanted so as to directly stick in the optic papilla in which optic nerve fibers concentrate, the optic nerve fibers serving to transfer pulse signals from retinal ganglion cells, and the indifferent electrode 22 is retained in the vitreous body of the eye. FIGS. 3 and 4 are views schematically showing an implanted state of those electrodes 21 and indifferent electrode 22. FIG. 3 is a horizontal sectional view of the entire eyeball and FIG. 4 is a view showing the optic papilla and its surrounding area. The signal wires 23 bundled by the tube 24, which connect the stimulation pulse signal generating unit 10 and the electrodes 21 (the indifferent electrode 22), are inserted in the eyeball through an opening formed in a sclera portion to extend along an inner wall (an eye fundus) of the eyeball up to the vicinity of the optic papilla, as illustrated in the figure. The electrodes 21 connected to the tips of the signal wires 23 extending out of the tube 24 are placed so as to individually stick by a predetermined depth into any positions of the optic papilla avoiding blood vessels. The indifferent electrode 22 is retained in the vitreous body of the eye. The tube 24 is fixed in the eye with tacks 25. In the present embodiment, the tube 24 is fixed in the eye with tacks but not limited thereto. It may be fixed with suturing or other fixing means or does not have to be fixed if unnecessary. In the present embodiment, further, the indifferent electrode 22 is retained in the vitreous body but not limited therein. One of the electrodes 21 placed in the optic papilla may be used as an indifferent electrode.

Using the electrical stimulation apparatus having the above structure, electrical stimulation is performed on the optic nerve. The present invention is arranged such that the electrodes are stuck in the optic papilla to output a predetermined electrical pulse signal, thereby improving vision of both of an operative eye and a fellow eye. The electrical stimulation pulse signal is preferably a signal capable of producing artificial vision called phosphene (light perception, photesthesia). Further, this electrical stimulation pulse signal is preferably a biphasic pulse signal. In this case, it is more preferable to output first a cathodic pulse signal and then output an anodic pulse signal. An electric current value (pulse height) for forming the electrical stimulation pulse signal is preferably 1 μA to 1 mA and more preferably 5 μA to 500 μA. A pulse width is preferably 10 μs to 1 ms and more preferably 50 μs to 600 μs. A repetition frequency is preferably about 1 Hz to about 1000 Hz and more preferably about 5 Hz to about 300 Hz. Cathodic and anodic pulses may be asymmetric or symmetric in waveform and equal (nearly equal) in the amount of charge per one pulse. Explaining in more detail, in the case where the pulse waveforms are asymmetric, one pulse waveform (e.g., on the cathode side) is formed in a rectangular pulse waveform based on the aforementioned condition while the other pulse waveform (e.g., on the anode side) is formed in a rectangular pulse waveform with half the current value and twice the pulse width of the former pulse waveform or with one fifth the current value and five times the pulse width of the former pulse waveform, or others. Setting such electrical stimulation pulse signal is performed by use of the aforementioned stimulation condition setting part 13. With a pulse signal output switch not shown, the electrical stimulation pulse signal is output from the electrodes implanted in the optic papilla. The apparatus of the present embodiment is arranged such that the electrodes are implanted to stick in the optic papilla to stimulate the optic nerve. Alternatively, the stimulating electrodes may be placed on a retina to electrically stimulate the cells constituting the retina under a predetermined stimulation condition in order to improve visual ability of an operative eye and a fellow eye.

Concrete cases are presented below, in which vision improvement was achieved by the electrical stimulation pulse signals from the electrodes placed in the optic papilla.

<Disease Cases>

The present evaluation was made in such a manner that the electrodes were stuck in any positions spaced at certain intervals, of the optic papilla of an examinee with vision loss from retinitis pigmentosa, and the electrical stimulation pulse signals were applied under different stimulation conditions, and then vision of the examinee was tested. In a vision test (a visual acuity test) conducted prior to the electrical stimulation, it was found that both the operative eye and the fellow eye had light perception vision.

Examinee: Adult male (Right eye). Implanted electrodes (three platinum wires each having a diameter of 50 μm were placed in any positions of the optic papilla). Indifferent electrode (a platinum wire having a diameter of 80 μm was retained in the vitreous body of the eye). The three placed electrodes are referred to as Electrode a, Electrode b, and Electrode c, respectively.

The placed Electrodes a, b, and c were caused to output the electrical stimulation pulse signals respectively to the examinee, thereby stimulating the optic nerve. Appearance of phosphenes was recorded. The stimulation condition of the electrical stimulation pulse signal was variously set in a range of an electrical current value of 5 μm to 150 μm, a pulse width of 80 μs to 580 μs, and a frequency of 2 Hz to 40 Hz. All the electrical stimulation pulse signals had biphasic waves. The biphasic wave was an asymmetric biphasic rectangular wave, and a negative (cathodic) pulse signal was first applied and then a positive (anodic) pulse signal was applied. To balance in charge with the waveform condition of the cathodic pulse, the waveform condition of the anodic pulse following the cathodic pulse was set so that the electrical current value was one fifth that of the cathodic pulse and the pulse width was five times that of the cathodic pulse.

The details and results of those stimulation conditions are shown in Table 1. The “number of tests” in the table represents that any electrode(s) are chosen from among the implanted electrodes (a to b) to output the electrical stimulation pulse signal(s) and this choice is counted as “1”.

TABLE 1
STIMULATION EVALUATION TEST

| | | | | Number of |
|---|---|---|---|---|
| Current | Pulse | | | appearance |
| Value | Width | Frequency | Number | of |
| (μA) | (μs) | (Hz) | of tests | phosphenes |
| 5 | 250 | 40 | 1 | 0 |
| 5 | 500 | 20 | 2 | 2 |
| 7 | 360 | 20 | 2 | 2 |
| 10 | 250 | 40 | 7 | 4 |
| 10 | 250 | 20 | 9 | 8 |
| 10 | 250 | 10 | 2 | 1 |
| 10 | 250 | 5 | 2 | 2 |
| 10 | 250 | 2 | 2 | 1 |
| 10 | 200 | 20 | 7 | 4 |
| 15 | 250 | 40 | 2 | 2 |
| 15 | 170 | 20 | 2 | 2 |
| 20 | 250 | 40 | 2 | 2 |
| 20 | 130 | 20 | 4 | 2 |
| 30 | 80 | 20 | 2 | 1 |
| 30 | 130 | 20 | 5 | 4 |
| 30 | 250 | 40 | 2 | 1 |
| 30 | 580 | 20 | 2 | 1 |
| 50 | 250 | 40 | 1 | 1 |
| 50 | 350 | 20 | 4 | 2 |
| 70 | 250 | 40 | 18 | 13 |
| 70 | 250 | 20 | 4 | 1 |
| 70 | 250 | 10 | 2 | 0 |
| 70 | 250 | 5 | 2 | 0 |
| 100 | 180 | 40 | 2 | 1 |
| 150 | 120 | 40 | 2 | 2 |

<Results>

In three months later after the stimulation evaluation tests, a visual acuity test was conducted on the operative eye (the right eye) and the fellow eye (the left eye). In the visual acuity test conducted three months later, it was proved that the operative eye could perceive hand movement, showing an improvement in vision. In the visual acuity test three months later, it was also found that the fellow eye could hand movement, showing an improvement vision.

What is claimed is:

1. An electrical stimulation method for improving vision of patient's eyes, comprising the following steps:
placing an electrode in one of the patient's eyes; and
outputting an electrical stimulation pulse signal from the electrode placed in the patient's eye under a predetermined stimulation condition to stimulate cells constituting a retina or an optic nerve to improve vision of the other patient's eye with no electrode placed.

2. The electrical stimulation method according to claim 1, wherein the step of placing the electrode in the patient's eye includes placing a needle-shaped electrode to stick in an optic papilla of the patient's eye.

3. The electrical stimulation method according to claim 1, wherein the step of outputting the electrical pulse signal from the electrode placed in the patient's eye under the predetermined stimulation condition to stimulate the cells constituting the retina or the optic nerve includes outputting the electrical stimulation pulse signal under a stimulation condition for allowing the patient to perceive a phosphene.

4. The electrical stimulation method according to claim 3, wherein the stimulation condition includes a combination of at least two chosen from an electrical current value, a frequency, and a pulse width.

5. The electrical stimulation method according to claim 4, wherein the electrical stimulation pulse signal is a biphasic wave stimulation pulse signal comprising a cathodic pulse signal and an anodic pulse signal.

6. The electrical stimulation method according to claim 5, wherein the cathodic and anodic pulse waveforms of the biphasic stimulation pulse signal are asymmetric or symmetric in waveform and equal in quantity of electrical charge of one pulse.

7. The electrical stimulation method according to claim 6, wherein the electrical current value is 1 HA to 1 mA.

8. The electrical stimulation method according to claim 6, wherein a repetition frequency of the stimulation pulse signal is 1 Hz to 1000 Hz.

9. The electrical stimulation method according to claim 6, wherein the pulse width of the stimulation pulse signal is 10 Us to 1 ms.

10. The electrical stimulation method according to claim 6, wherein the cathodic pulse signal is first output and then the anodic pulse signal is output.
